(19) United States
(12) Patent Application Publication
(10) Pub. No.: US 2008/0096794 A1
(43) Pub. Date: Apr. 24, 2008

(54) Peptide Mixture From Peptides Having A Molecular Weight Of from 1000 To 5000 Dalton

(76) Inventors: Gunther Boehm, Echzell (DE); Christopher Beermann, Neu-Anspach (DE); Bernd Stahl, Rosbach (DE); Gilda Georgi, Friedrichsdorf (DE); Joachim Schmitt, Hosbach (DE); Nadine Bock, Bremen (DE); Sorge Kelm, Lilienthal (DE)

Correspondence Address: BACON & THOMAS, PLLC, 625 SLATERS LANE, FOURTH FLOOR, ALEXANDRIA, VA, 22314, US

(21) Appl. No.: 11/660,595

(22) PCT Filed: Aug. 12, 2005
(86) PCT No.: PCT/EP05/08805

§ 371 Date: Feb. 20, 2007

(30) Foreign Application Priority Data
Aug. 20, 2004 (DE) 10 2004 040 452.6

(51) Int. Cl. A61K 38/00; A23C 9/00; A23J 3/08; A61P 43/00; C07K 16/00; A23J 3/10; A23L 1/305

(52) U.S. Cl. 514/2; 426/42; 426/656; 530/300

(57) ABSTRACT

The invention relates to the use of a peptide mixture comprising peptides with a molecular weight from 1000 to 5000 Daltons for the prevention and/or treatment of tumour diseases, of diseases which are associated with a development disorder of the immune system, of diseases of the immune system, of autoimmune reactions, of allergies and of inflammation in man, a protein component which contains such a peptide mixture and a formula food containing this peptide mixture. The peptides are preferably obtained from non-human proteins suitable for nutritional purposes, in particular by the enzymatic or hydrolytic cleavage of these proteins.

The invention relates to the use of a peptide mixture comprising peptides with a molecular weight from 1000 to 5000 Daltons for the prevention and/or treatment of tumour diseases, of diseases which are associated with a developmental disorder of the immune system, of diseases of the immune system, of autoimmune reactions, of allergies and of inflammation in man, a protein component which contains such a peptide mixture and a formula food containing this peptide mixture.

Many formula foods which consist of artificial foods which are formulated on the basis of non-human components have already been proposed. Such formula foods include for example baby, child and adult foods.

For the production of such formula foods, proteins from other species are used. These consist for example of proteins from cattle. In most cases, the caseins and whey proteins of cow's milk are used.

Also known is the use of proteins from plants or of microbial origin for the production of such formula foods. These proteins for example include soya proteins.

In the feeding of such formula foods or of substitute foods based on cow's milk to babies, problems often arise, which for example take the form of an allergy. This applies even for adapted baby foods and baby milk foods which are matched as closely as possible to human milk, which, as is well known, is best for the baby.

In order to reduce the occurrence for example of allergies, formula foods based on hydrolysed proteins, for example hydrolysed caseins or whey proteins, are already on sale. Soya proteins have also already found their way into such formula foods.

However, both for the intact proteins and also for the hydrolysed proteins, from non-human sources, the fact remains that these differ from those of humans and human milk. These differences relate inter alia to the proportions of the various amino acids, the sequence of the amino acids in the proteins, the degree of sialisation of the proteins and the cleavage sites of the proteins for various proteases. Because of the different cleavage sites, different residual chains of amino acids, which are described as peptides, are formed in the cleavage (digestion) of non-human proteins and the digestion of proteins from human milk. On the one hand, such proteins are degraded by the human and animal body to single amino acids which are then absorbed and metabolised by the body. However, certain peptides are also absorbed by the body without further cleavage and can then also exhibit regulatory effects in the human body.

The object of the present invention is to demonstrate a way in which the inherent disadvantages with the previously used proteins and peptides can be avoided.

This object is solved by the teaching of the claims.

Namely, it has surprisingly been found that the actions of peptides from human proteins are different from the actions of non-human peptides. In fact, through the use of a mixture of certain peptides, the activity of B lymphocytes in man can be attenuated.

Thus according to the invention a peptide mixture comprising non-human peptides with a molecular weight from 1000 to 5000 Daltons and preferably from 1000 to 3000 Daltons is used. The stated range from 1000 to 5000 Daltons here includes all smaller ranges lying within this larger range, for example 1500 to 5000, 1500 to 4500, 1500 to 4000, 2000 to 4000, 2500 to 4000, 1000 to 4000, 1000 to 3500 and 1500 to 3500, to name just a few smaller ranges. This list could be extended at will. Preferably the peptides used according to the invention have a molecular weight from 1000 to 3000 Daltons.

These peptides are preferably obtained from non-human proteins suitable for nutritional purposes. Thus peptides can be used which were obtained from animal proteins, for example from cattle, camels, goats or sheep and the respective milks including the caseins and whey proteins of these animals. Apart from this, proteins from plants, for example soya protein, rice protein, rape protein, lupin protein, yeast protein and pea protein can be used. Even proteins from microbial sources can be used.

These non-human proteins used as raw materials are hydrolytically or enzymatically cleaved by standard methods. The mixtures of protein fragments or peptides resulting from this are then separated by filtration or chromatographic separation processes in such a manner that a fraction with peptides from 1000 to 5000 Daltons in size is obtained. It is also possible to proceed in such a manner that the size range obtained is smaller than the this larger range, as described above.

Surprisingly, these peptides obtained from non-human sources have a different action on the immune system in man than the human peptides of comparable size which are obtained when proteins for example from human milk are digested.

According to a preferred embodiment, the proteins used or the peptides obtained after the cleavage thereof are at least partially desialised. In other words, the sialic acid groups bound to the proteins or to the peptides are at least partially cleaved off. For example, only a few or even all sialic acid groups can be removed. Preferably, 50 to 100% of the sialic acid groups present are removed. The effects described above can be intensified by such removal of the sialic acid groups.

The peptide mixture used according to the invention can be consumed directly by humans, for example in the form of a food supplement. This mixture can however also be incorporated into other food products. These include in particular clinical foods and formula foods, in particular for babies and small children, and among these more particularly baby foods and baby milk foods.

Thus a peptide mixture comprising peptides with a molecular weight between 1000 and 5000 Daltons is also a subject matter of the invention. The peptide mixture is preferably obtained by hydrolysis or enzymatic cleavage of a natural, non-human protein component. Long-chain and short-chain peptides are then removed, preferably by single or double ultrafiltration.

Further, according to the invention a clinical food and a formula food, preferably a baby food or infant formula, respectively, is provided, which contains a peptide mixture according to the invention. It has in fact emerged that this peptide mixture according to the invention advantageously influences the immune system of a child and in particular of a baby/infant. The formula food according to the invention is preferably enriched with the peptides used according to the invention. According to a preferred embodiment, a baby food is thus provided, which contains a fat component, a carbohydrate component and a protein component. The protein component thus contains the peptide mixture according to the invention. In addition, other components can be present, which can usually be among the protein components suitable for nutritional purposes, for example peptides which are not part of the peptide mixture according to the invention, free amino acids and proteins.

The expression protein component used here thus refers to the totality of the proteins, peptides and free amino acids. Such a protein component can thus contain intact proteins, a peptide mixture according to the invention of the type described above and also peptides which are not part of the peptide mixture according to the invention, and also free amino acids. Here, all protein and protein components are preferably of non-human origin.

Also a subject matter of the invention is a formula food, in particular a baby food or infant formula, respectively, which contains a fat component, a carbohydrate component and a protein component, wherein the protein component contains at least 10 wt. % peptides with a molecular weight of 1000 to 5000 Daltons, based on the total weight of the protein component, preferably at least 25 wt. %, more preferably at least 50 wt. %, more preferably at least 75 wt. %, still more preferably at least 90 wt. % and most preferably at least 95 wt. %. Preferably, the protein component contains at least 10 wt. % peptides with a molecular weight from 1000 to 3000 Daltons, based on the total weight of the protein component, preferably at least 25 wt. %, more preferably at least 50 wt. %, more preferably at least 75 wt. %, still more preferably at least 90 wt. % and most preferably at least 95 wt. %. The expression protein component or protein used here relates to the totality of the proteins, peptides and free amino acids.

The formula food, in particular baby food or infant formula, respectively, according to the invention contains less than 10 wt. % free amino acids, based on the total weight of protein, more preferably less than 5 wt. % and most preferably less than 1 wt. %. The content of protein molecules of intermediate chain length is also restricted. Preferably the formula food according to the invention contains less than 10 wt. % peptides with a molecular weight between 5000 and 7500 Dalton, based on the total protein, more preferably less than 5 wt. % and most preferably less than 1 wt. %. Natural and biologically active proteins can also be added to the formula food or composition according to the invention.

The formula food according to the invention is preferably used as baby food or as infant formula and preferably contains 7.5 to 12.5 energy % protein, 40 to 55 energy % carbohydrates and 35 to 50 energy % (en. %) fat.

Digestive disorders (for example hard stools, insufficient stool volume, diarrhea) are a major problem with many babies, and also with sick people, who are taking liquid foods. It has been found that such digestive disorders can be decreased in that the composition according to the invention can be reduced into the form of a liquid food which has an osmolality from 50 to 500 nOsm/kg and particularly preferably from 100 to 400 mOsm/kg.

In view of the aforesaid, it is also important that the liquid food does not have an excessive caloric density, but still provides a sufficient quantity of calories for the nutrition of the person. The liquid food therefore preferably has a caloric density from 0.1 to 2.5 kcal/ml, more preferably between 0.5 and 1.5 kcal/ml, and most preferably between 0.6 and 0.8 kcal/ml.

The formula food according to the invention typically contains fats, so that it can serve for nutritional purposes. The quantity of the saturated fatty acids here is preferably less than 58 wt. %, based on the total quantity of fatty acids and more preferably less than 45 wt. %. The concentration of the singly unsaturated fatty acids is preferably 17 to 60%, based on the weight of the total fatty acids. The concentration of multiply unsaturated fatty acids in the formula food according to the invention is preferably 11 to 36%, based on the weight of the total fatty acids. The fats are essential for the growth of the child or the baby. In particular, the multiply unsaturated fats further the development of the immune system and thus have a synergistic action with the protein mixture according to the invention.

The formula food according to the invention preferably contains 0.3 to 1.5 g of linoleic acid (LA) per 100 ml of the liquid food ready for administration or ready for feeding and at least 50 mg of linolenic acid (ALA) per 100 ml. The formula food according to the invention preferably contains 1.8 to 12.0 wt. % LA, based on the dry weight of the food, and at least 0.30 wt. % ALA, based on the dry weight of the food. The LA/ALA weight ratio is preferably 5 to 15. In a much preferred embodiment, the formula food according to the invention contains long-chain highly unsaturated fatty acids (long-chain polyunsaturated fatty acids; LC-PUFA), since these are particularly effective for the development of the immune system and for the prevention of allergies and infections. It has surprisingly been found that various LC-PUFA advantageously influence the barrier integrity. The formula food according to the invention therefore contains at least one such LC-PUFA fatty acid selected from the group consisting of eicosapentaenoic acid (EPA), docosahexaenoic acid (DHA) and arachidonic acid (AA). The food according to the invention preferably contains EPA, DHA and AA together. A fat mixture with the properties described above has a synergistic action with the peptide mixture of hydrolysed proteins according to the invention with regard to the prevention and/or treatment of diseases, allergies and infections which are connected with the immune system.

The formula food according to the invention contains 2.1 to 6.5 g fat per 100 ml when it is in the liquid form ready for administration or ready for feeding. The formula food preferably contains, based on the dry weight, 12.5 to 30 wt. % fat. The food according to the invention preferably contains 35 to 60 en. % fat and in particular 39 to 50 en. % fat.

In order further to increase the effectiveness of the formula food and protein mixture according to the invention, the protein mixture according to the invention is preferably combined with acidic oligosaccharides. These acidic oligosaccharides reduce the adhesion of pathogenic germs, substances, etc. to the intestinal epithelium and thereby reduce the occurrence of intestinal stress and immunological stress. Owing to the fact that these stresses are reduced, uniform and optimal development of the immune system is ensured. Hence the peptide mixture according to the invention and the acidic oligosaccharides have a synergistic action in this respect.

The expression “acidic oligosaccharides” relates to oligosaccharides which contain at least one acid group selected from the group consisting of N-acetylneuraminic acid, N-glycoloylneuramic acid, free or esterified carboxylic acid groups, a sulphuric acid group and a phosphoric acid. The acidic oligosaccharide is preferably a polyhexose. Preferably at least one of the aforesaid acid groups is located on the terminal hexose unit of the acidic oligosaccharide. Preferably the acidic oligosaccharide has the formula shown in FIG. 1, wherein the terminal hexose unit (on the left) preferably has a double bond. The acidic oligosaccharide according to the invention is most preferably a pectin hydrolysate. Preferably the acidic oligosaccharide contains a carboxylic acid group on the terminal hexose unit, and this carboxylic acid can be free or esterified. Processes for the production of esterified pectin hydrolysates which can be used according to the invention are described in the PCT patent applications WO 01/60378 and/or WO 02/42484, the content whereof is hereby expressly made content of the present application.

The hexose units which are not the terminal hexose unit(s) are preferably uronic acid units, more preferably galacturonic acid units. The carboxylic acid groups on these units can be free or at least partially esterified and are preferably at least 10% methylated (see below).

[image]

For the formula reproduced above, the following applies:

The residue R is preferably selected from the group consisting of hydrogen, hydroxy and an acid group, preferably hydroxy,

at least one of the residues R₂, R₃, R₄ and R₅ stands for N-acetyl-neuraminic acid, N-glycoloylneuraminic acid, a free or esterified carboxylic acid group, a sulphuric acid group or a phosphoric acid group and the remaining residues R₂, R₃, R₄ and R₅ stand for hydroxy and/or hydrogen, wherein one of the residues R₂, R₃, R₄ and R₅ preferably stands for N-acetylneuraminic acid, N-glycoloylneuraminic acid, a free or esterified carboxylic acid group, a sulphuric acid group or a phosphoric acid group and the remaining residues stand for hydroxy and/or hydrogen, and wherein most preferably one of the residues R₂, R₃, R₄ and R₅ means a free or esterified carboxylic acid group and the remaining residues R₂, R₃, R₄ and R₅ stand for hydroxy and/or hydrogen, and

n means a whole number and stands for the number of hexose units (compare also the degree of polymerisation discussed below), where these can be any desired hexose unit; n preferably stands for a whole number from 1 to 5000.

The hexose unit or hexose units is/are preferably uronic acid unit(s).

More preferably, the following applies: R₁, R₂ and R₃ stand for hydroxy, R₄ stands for hydrogen, R₅ stands for a carboxylic acid group, n stands for any number between 1 to 250, preferably between 1 to 10, and the hexose unit is galacturonic acid.

The acidic oligosaccharide has one, preferably two, terminal uronic acid units, which can be present in free form or in esterified form. The terminal uronic acid unit is preferably selected from the group consisting of galacturonic acid, glucuronic acid, guluronic acid, iduronic acid, mannuronic acid, riburonic acid and altruronic acid. These units can be present in free or esterified form. In a more preferable embodiment, the terminal hexose unit has a double bond which is preferably located between the C₄ and the C₅ atom of the terminal hexose unit. Preferably one of the terminal hexose units has the double bond. The terminal hexose (for example uronic acid) preferably has the structure/formula shown in FIG. 2 below.

[image]

For the above formula, the following applies.

The residue R is preferably selected from the group consisting of hydrogen, hydroxy and an acid group, preferably hydroxy, and

at least one of the residues R₂, R₃, R₄ and R₅ stands for N-acetyl-neuraminic acid, N-glycoloylneuraminic acid, a free or esterified carboxylic acid group, a sulphuric acid group or a phosphoric acid group and the remaining residues R₂, R₃, R₄ and R₅, stand for hydroxy and/or hydrogen. Preferably, one of the residues R₂, R₃, R₄ and R₅ stands for N-acetylneuraminic acid, N-glycoloylneuraminic acid, a free or esterified carboxylic acid group, a sulphuric acid group or a phosphoric acid group and the remaining residues R₂, R₃, R₄ and R₅ stand for hydroxy and/or hydrogen. More preferably, one of the residues R₂, R₃, R₄ and R₅ stands for a free or esterified carboxylic acid group and the remaining residues R₂, R₃, R₄ and R₅ stand for hydroxy and/or hydrogen.

n stands for a whole number and refers to the number of hexose units (compare also the degree of polymerisation discussed below), where these can be any desired hexose unit. n advantageously means a whole number between 1 and 5000 and stands for the number of hexose units, where these hexose units preferably represent uronic acid units and more preferably galacturonic acid units. The carboxylic acid groups on these units can be present in free or in partially esterified form and preferably at least partially methylated.

More preferably, R₂ and R₃ stand for hydroxy, R₄ for hydrogen and R₅ for a free or esterified carboxylic acid group.

The residues R, R₂, R₃, R₄ and R₅ and the number n for the formula shown in FIG. 2 preferably have the same meanings as the corresponding residues or the corresponding number in the formula shown in FIG. 1.

In a further embodiment, a mixture of acidic oligosaccharides is used, which have a different DP and/or contain both unsaturated and also saturated terminal hexose units. Preferably at least 5%, more preferably at least 10% and most preferably at least 25% of the terminal hexose units of the acidic oligosaccharide are unsaturated hexose units (compare FIG. 2). Since each individual acidic oligosaccharide preferably contains only one unsaturated terminal hexose unit, preferably not more than 50% of the terminal hexose units are unsaturated hexose units (i.e. contain a double bond).

The mixture of acidic oligosaccharides preferably contains 2 to 50%, preferably 10 to 40% of unsaturated hexose units, based on the total number of hexose units,

The acidic oligosaccharides used according to the invention have a degree of polymerisation (DP) from 1 to 5000 and preferably from 1 to 1000, more preferably from 2 to 250, more preferably from 2 to 50 and most preferably from 2 to 10. If a mixture of acidic oligosaccharides of differing degree of polymerisation is used, then the average degree of polymerisation of the acidic oligosaccharide mixture is preferably between 2 and 1000, more preferably between 3 and 350 and most preferably between 3 and 50. In this respect, compare also FIG. 1, wherein the sum of “n” and the terminal unit (i.e. n+1) represents the degree of polymerisation.

It has been found that a low degree of polymerisation of the oligosaccharides improves the palatability and results in decreased product viscosity, if the acidic oligosaccharide is administered in liquid form. The acidic oligosaccharide can be a homogeneous or a heterogeneous carbohydrate.

The acidic oligosaccharides preferably have a methoxylation level of more than 20%, preferably more than 50% and most preferably of more than 70%. Preferably the acidic oligosaccharides have a methylation level of more than 20%, preferably of more than 50% and most preferably of more than 70%.

The acidic oligosaccharide, which is preferably a pectin hydrolysate, is preferably administered in a quantity from 10 mg to 100 g per day, preferably from 100 mg to 50 g per day and most preferably from 0.5 to 20 g per day.

Hence other peptides and/or proteins can also be present in these foods and food products as well as the peptide mixture according to the invention. Further, a fat component and a carbohydrate is preferably also present as well as vitamins, minerals and trace elements.

The peptide mixture used according to the invention can also be formulated in the form of a medicament. Hence the use of the peptide mixture described here for the production of a food and a pharmaceutical composition for the reduction of the activity of B lymphocytes in man is also an subject matter of the invention. Such a pharmaceutical composition or medicament can consist of a peptide mixture described here alone. Normal carriers, adjuvants and/or other components used in pharmaceutical compositions can also be present. One or several other pharmaceutically active substance(s) can also be present. The food and the pharmaceutical composition are in particular administered enterally or orally.

The peptide mixture according to the invention is used in particular for the prevention and/or treatment of tumour diseases, for the prevention and/or for the treatment of diseases which are associated with a developmental disorder of the immune system, for the prevention and/or for the treatment of diseases of the immune system, for the prevention and/or for the treatment of autoimmune reactions, for the prevention and/or for the treatment of allergies and for the prevention and/or for the treatment of inflammation.

The invention is illustrated below by means of further examples for the production of a peptide mixture according to the invention. Here, unless otherwise stated, % values refer to the weight.

### EXAMPLE 1

### Production of a Baby Food with Hydrolysed Bovine Peptides

140 kg of casein (90% protein), are dissolved in warm water at 60° C. After a pasteurisation step, the preparation is cooled to 40° C. and trypsin/chymotrypsin (enzyme:substrate ratio of 1:280) is added. The solution is incubated for 2.5 hours at 45° C. After deactivation of the enzyme at 90° C. for 10 minutes, a two-stage ultrafiltration is performed. 1^{st} Stage: ultrafiltration of the hydrolysate solution with a cut-off of 3000 Daltons; 2^{nd} Stage: ultrafiltration of the permeate from the first stage with a cut-off of 1000 Daltons. 290 kg of whey powder (13% protein), 67 kg of whey protein concentrate (76% protein), 154 kg of lactose, 49 kg of maltodextrins, 285 kg of a suitable lipid mixture and the quantities of minerals, trace elements and vitamins recommended for baby foods are added successively to the casein peptides now enriched in the retained liquid. After complete dissolution of all components, the solution is homogenised, pasteurised and evaporated to a dry weight content of 35-45%. As the final step, a spray-drying is performed.

### EXAMPLE 2

### Production of a Peptide Mixture Modulating the Immune System as a Supplement for Foods or as a Supplement in Capsule or Sachet Form

Caseinate, obtained from bovine milk, is dissolved at a concentration of 10% in warm water at 60° C. and the solution pasteurised. After cooling of the solution to 45° C., the pH value is adjusted to 7.2 with dilute sodium hydroxide solution. Trypsin is then added (enzyme:substrate ratio of 1:150) and the solution incubated for 180 minutes at 45° C. Next, the same quantity of chymotrypsin is added and the solution incubated for a further 30 mins at the same temperature. After termination of the hydrolysis, the solution is heated for 10 minutes at 90° C. to deactivate the enzymes and then spray- or freeze-dried. The majority of the peptides thus obtained are between 1000 Daltons and 3000 Daltons in size and can be used thus as a supplement in capsule or sachet form.

### EXAMPLE 3

### Production of a Peptide Mixture from a Mixture of Soya and Rice Proteins as a Supplement for Foods

Soya and rice proteins are mixed in a ratio of 60 to 40. Next this mixture is dissolved (suspended) at a protein concentration of 6-10% in warm water at 45° C. and the solution pasteurised. After cooling of the solution to 45° C., the pH value is adjusted to 7.0 with dilute sodium hydroxide solution and a mixture of trypsin and chymotrypsin (1:1) added with an enzyme:substrate ratio of 1:200 and the solution incubated for 180 mins at 45° C. The pH value is checked at intervals of ca. 15 mins and if necessary again adjusted to 7.0. After termination of the hydrolysis, the solution is heated for 10 minutes at 90° C. to deactivate the enzymes. The peptides are freeze- or spray-dried and can be used thus as supplements for immunomodulating foods.

### EXAMPLE 4

### Chemical Desialisation of Proteins and Peptides

Intact proteins or peptides obtained by enzymatic or chemical hydrolysis are dissolved at a concentration of 10% in warm water at 60° C. and the solution pasteurised. After cooling of the solution to 45° C., the pH value is adjusted to 1.75 with 0.18 n hydrochloric acid (HCl). The solution is then incubated and stirred for 8 hours at 50° C. or 5 hours at 60° C. or 3 hours at 70° C. Next the solution is adjusted to pH 6.6 to pH 6.8 with KOH or NaOH. The desialised proteins or peptides thus obtained can be further processed as appropriate for the production of formulations according to the invention.

### EXAMPLE 5

### Enzymatic Desialisation of Proteins and Peptides

Intact proteins or peptides obtained by enzymatic or chemical hydrolysis are dissolved at a concentration of 10% in warm water at 60° C. and the solution pasteurised. After cooling of the solution to 45° C., the pH value is adjusted to 7.2 with dilute sodium hydroxide solution. A commercially available enzyme which cleaves the sialic acids from proteins and peptides non-specifically is added. This so-called non-specific sialidase cleaves 2-3, 2-6 and 2-8 linked sialic acids from the proteins and peptides. The solution is incubated for 24 hours at 25° C. After termination of the desialisation, the solution is heated for 10 minutes at 90° C. to deactivate the enzyme and then cooled again to room temperature. The solution of desialised proteins and/or peptides thus obtained can be further processed for the production of appropriate formulations according to the invention.

1. Use of a peptide mixture comprising peptides with a molecular weight from 1000 to 5000 Daltons, which have been obtained by enzymatic or hydrolytic cleavage of non-human proteins suitable for nutritional purposes, for the prevention and/or treatment of tumour diseases, of diseases which are associated with a developmental disorder of the immune system, of diseases of the immune system, of autoimmune reactions, of allergies and of inflammation in man or for the production of a formula food, a supplement for a normal food or a pharmaceutical composition for the prevention and/or treatment of tumour diseases, of diseases which are associated with a developmental disorder of the immune system, of diseases of the immune system, of autoimmune reactions, of allergies and of inflammation.

2. Use according to claim 1, characterised in that the peptides have been obtained from proteins which are selected from the group consisting of animal proteins from cattle, camels, goats or sheep and from the respective milks including the caseins and whey proteins of these animals, soya protein, rice protein, rape protein, lupin protein, yeast protein and pea protein.

3. Use according to claim 1, characterised in that the proteins used or the peptides obtained therefrom have been at least partially desialised.

4. Use according to claim 1, characterised in that the formula food is a baby food or a baby milk food.

5. Use of a peptide mixture comprising peptides with a molecular weight from 1000 to 5000 Daltons for the prevention and/or treatment of tumour diseases, of diseases which are associated with a developmental disorder of the immune system, of diseases of the immune system, of autoimmune reactions, of allergies and of inflammation in man or for the production of a formula food, a supplement for a normal food or a pharmaceutical composition for the prevention and/or treatment of tumour diseases, of diseases which are associated with a developmental disorder of the immune system, of diseases of the immune system, of autoimmune reactions, of allergies and of inflammation.

6. Use according to claim 1, characterised in that the peptide mixture is used together with oligosaccharides which have at least one acidic group which is selected from the group consisting of N-acetylneuraminic acid, N-glycoloylneuraminic acid, a free or esterified carboxylic acid group, a sulphuric acid group and a phosphoric acid group.

7. Formula food with a peptide fraction containing peptides with a molecular weight from 1000 to 5000 Daltons, less than 10 wt. % free amino acids, based on the total weight of the protein, and less than 10 wt. % peptides with a molecular weight between 5000 and 100000 Daltons, based on the total weight of the protein, and a protein content of at least 75 wt. %, based on the total dry weight of the peptide fraction.

8. Formula food with 7.5 to 12.5 en. % protein, 40 to 55 en. % carbohydrate and 35 to 50 en. % fat and containing a protein component which contains at least 10 wt. % peptides with a molecular weight of 10000 to 5000 Daltons, based on the total weight of the protein component.

9. Formula food according to claim 8, characterised in that the protein component contains less than 10 wt. % free amino acids, based on the total weight of the protein.

10. Use of a formula food according to claim 7, for the production of a composition for the treatment of diseases and disorders of the immune system, in particular of allergies and infections.

11. Method for the prevention and/or treatment of tumour diseases, of diseases which are associated with a developmental disorder of the immune system, of diseases of the immune system, of autoimmune reactions, of allergies and of inflammation in man, characterised in that a peptide mixture comprising peptides with a molecular weight from 1000 to 5000 Daltons, which have been obtained by enzymatic or hydrolytic cleavage of non-human proteins suitable for nutritional purposes, is administered.

12. Method according to claim 10, wherein the peptides with a molecular weight from 1000 to 5000 Daltons have been obtained by enzymatic or hydrolytic cleavage of non-human proteins suitable for nutritional purposes.

13. Use according to claim 2, characterised in that the proteins used or the peptides obtained therefrom have been at least partially desialised.

14. Use of a formula food according to claim 8, for the production of a composition for the treatment of diseases and disorders of the immune system, in particular of allergies and infections.

15. Use of a formula food according to claim 9, for the production of a composition for the treatment of diseases and disorders of the immune system, in particular of allergies and infections.
